(19) United States
(12) Patent Application Publication
(10) Pub. No.: US 2015/0313570 A1
(43) Pub. Date: Nov. 5, 2015

(54) OPERATION SWITCH FOR USE ON X-RAY SYSTEM, AND X-RAY SYSTEM INCLUDING SAME

(71) Applicant: Yonghae HAN, (US)

(72) Inventor: Yonghae Han, Gangwon-do (KR)

(21) Appl. No.: 14/649,346

(22) PCT Filed: Nov. 25, 2013
(86) PCT No.: PCT/KR2013/010718

§ 371 Date: Jun. 3, 2015

(30) Foreign Application Priority Data
Dec. 3, 2012 (KR) 20-2012-0011201
Dec. 11, 2012 (KR) 20-2012-0011614
Apr. 3, 2013 (KR) 20-2013-0002461

(51) Int. Cl. A61B 6/00; H01H 36/00; H01H 13/50

(52) U.S. Cl. CPC A61B 6/548; H01H 13/50; H01H 36/008; A61B 6/4283

(57) ABSTRACT

The present invention relates to an operation switch for use on an X-ray system and to an X-ray system including same. The operation switch, which includes a button part for operating a generator for driving an X-ray tube, includes: a switch body part; a link part which is formed so as to extend integrally with the button part and which is slid along the outer side surface of the switch body part when the button part is under a pressing operation; a switching means for being switched when the link part slides; and a transmission means for generating and transmitting a signal based on the switching operation of the switching means. A controller controls an X-ray receiving cassette to be ON when the signal is received. Accordingly, the convenience of X-ray imaging can be improved when using the X-ray system.

### TECHNICAL FIELD

The present invention relates to an operation switch for an X-ray system and a X-ray system including the same that are used in a medical institution, and more particularly, relates to an operation switch for an X-ray system and a X-ray system including the same that are capable of improving convenience of X-ray imaging.

### BACKGROUND ART

FIG. 1 shows an X-ray system having an old system specification of the related art, and an X-ray system 10 includes an X-ray reception cassette 13 that is attached to an X-ray stand 11 or an X-ray bed 12, an X-ray tube 14 that emits X-rays to the X-ray reception cassette 13, a generator 15 for driving the X-ray tube, and an operation switch 16 that is provided with a button part 16a for operating the generator.

Accordingly, when a user presses the button part 16a of the operation switch 16, the X-ray tube 14 is driven by the control of the generator 15 to emit X-rays. The emitted X-rays pass through a patient, and are received by the X-ray reception cassette 13 attached to the X-ray stand 11 or the X-ray bed 12.

The X-ray reception cassette 13 having the old system specification is an X-ray film, and an image received by the X-ray reception cassette is obtained in a photograph printing method using a developer.

Meanwhile, in an improved X-ray system having an old system specification, the X-ray reception cassette 13 includes a computed radiography (CR) cassette, and when the X-rays are received by the CR cassette, an image is obtained by extracting the X-rays and being read by a reader that is separately provided.

However, according to the X-ray system 10 having the old system specification of the related art, there is a problem in that the image needs to be developed for performing photograph printing after the X-ray reception cassette 13 that receives the X-rays, that is, the X-ray film or the CR cassette is separately detached, or the image needs to be read by the separate reader.

Accordingly, in recent years, in order to solve the problem of the X-ray system 10 having the old system specification of the related art, an X-ray system 20 having a new system specification using a direct-capture radiography (DR) cassette shown in FIG. 2 has been introduced.

The X-ray system 20 includes an X-ray reception cassette 23 which is a DR detector attached to the X-ray stand (see reference numeral 11 of FIG. 1) or the X-ray bed (see reference numeral 12 of FIG. 1), an X-ray tube 24 for emitting X-rays to the X-ray reception cassette 23, a generator 25 for driving the X-ray tube, an operation switch 26 provided with a button part 26a for operating the generator, an image processing and displaying device 27 for displaying reception data of the X-ray reception cassette, as an image, and a DR controller 28 for controlling the X-ray reception cassette and the image processing and displaying device.

According to the X-ray system 20 having the new system specification, the DR controller 28 electrically connected to the DR detector 23 that receives the X-rays obtains data, and the obtained data is transmitted to the image processing and displaying device 27 (which is practically the same as a computer system), and an image is immediately output. Thus, it may be possible to quickly and conveniently obtain the image.

However, since the entire system is expensive, the x-ray system 20 having such a new system specification upgraded by adding the DR detector 23, the DR controller 28 and the image processing and displaying device 27 to the X-ray system 10 having the old system specification of the related art has been practically used.

However, as shown in FIG. 2, the DR controller 28 controls the DR detector 23 by receiving an operation signal of the button part 26a of the operation switch 26 and interworking with the generator 25. Thus, an electric wire 29 extracted from the operation switch 26 is branched into two wires 29a and 29b and the branched wires are respectively connected to the generator 25 and the DR detector 23. For this reason, in the X-ray system 10 having the old system specification shown in FIG. 1 there is a problem in that a separate extract line (not shown) needs to be provided on the electric wire 19 extracted from the switch 26 and the extract line needs to be connected to the added DR controller.

The addition of the extract line is likely to be illegal in the regulations of Korean Food and Drugs Administration. Since the addition of the extract line may be cause of exemption in arguing the defect of medical equipment when a trouble occurs, an improved system is needed to solve the problem.

As shown in FIG. 3, in the X-ray system 10 having the old system specification or the X-ray system 20 having the new system specification, an X-ray stand 11 is provided at one wall within an X-ray room 1, an X-ray bed 12 is provided in front of the X-ray stand, and an X-ray tube 14 is provided at an upper ceiling of the bed 12. With such a configuration, X-ray imaging is performed on a patient. The X-ray operation switch 16 or 26 is operated by an operator in an operation room 2 provided as a separate space within the X-ray room 1, and thus, the X-ray imaging is performed. In this case, in order to prevent X-ray exposure of the operator in the X-ray imaging, a window 3 including a lead component is provided.

However, in the structure in which the X-ray system 10 or 20 is provided within the X-ray room 1, when the operator requests that the patient holds his or her breath in X-ray imaging or the operator notifies that the X-ray imaging is completed, such communication may not be easily performed between the operator and the patient. For this reason, by opening a door 4 of the operation room 2 when the X-ray system is actually operated, voice is allowed to be easily transmitted to the patient. However, the X-rays are transmitted to the operator through the opened door, and the operator is exposed to the X-rays. Thus, there is a problem in that the window 3 for preventing the exposure of the operator to the X-rays is not useful.

### DISCLOSURE

### Technical Problem

An object of the present invention is to provide an operation switch for an X-ray system and an X-ray system including the same that are capable of legally upgrading the old system specification of the related art easily and conveniently.

Another object of the present invention is to provide an X-ray system capable of preventing the exposure of an operator to X-rays when X-ray imaging is operated in an X-ray room.

### Technical Solution

In order to achieve the above object, the present invention provides an X-ray system that includes an X-ray reception cassette which is attached to an X-ray stand or an X-ray bed, an X-ray tube which emits X-rays to the X-ray reception cassette, a generator which drives the X-ray tube, an operation switch that is provided with a button part for operating the generator, an image processing and displaying device which displays reception data of the X-ray reception cassette as an image, and a controller which controls the X-ray reception cassette and the image processing and displaying device. The operation switch includes a switch body, a link part that is integrally formed with the button part so as to extend, and slides on an outer surface of the switch body at the time of pressing the button part, switching means for performing a switching operation according to the sliding of the link part, and transmission means for generating a signal according to the switching operation of the switching means, and for transmitting the generated signal to the controller. The controller performs control such that the X-ray reception cassette is turned on when the signal is received.

In order to achieve the above object, the present invention provides an operation switch for an X-ray system including a switch body that is provided with a button part at one end thereof; a link part that is integrally formed with the button part so as to extend, and slides on an outer surface of the switch body when the button part is pressed; switching means that is switched according to the sliding of the link part; and transmission means for generating a signal according to the switching operation of the switching means, and for transmitting the generated signal.

In order to achieve the above object, the x-ray system according to the present invention provides an X-ray system further includes a speaker control unit that is operated by receiving a signal of the transmission means; and a speaker that is disposed adjacent to the X-ray stand or the X-ray bed, and outputs a voice signal for requesting patient's cooperation in X-ray imaging under the control of the speaker control unit.

In order to achieve the above object, the present invention provides an X-ray system that includes an X-ray reception cassette which is a DR detector attached to an X-ray stand or an X-ray bed, an X-ray tube which emits X-rays to the X-ray reception cassette, a generator which drives the X-ray tube, an operation switch which is provided with a button part for operating the generator, an image processing and displaying device which displays reception data of the X-ray reception cassette as an image, and a DR controller which controls the X-ray reception cassette and the image processing and displaying device, the DR controller performing control such that the X-ray reception cassette is turned on when a signal generated according to the switching operation of the operation switch is received. The X-ray system includes: a speaker control unit that is operated by the DR controller by turning on the X-ray reception cassette; and a speaker that is disposed adjacent to the X-ray stand or the X-ray bed, and outputs a voice signal for requesting patient's cooperation in X-ray imaging under the control of the speaker control unit.

### Effect of the Invention

As described above, according to the operation switch for an X-ray system and the X-ray system including the same according to the present invention, since the operation switch includes the separate transmission means for generating and transmitting the signal, the signal is received by the controller simultaneously when the operation switch is operated. Thus, the generator and the controller can perform control in an interworking manner, so that it is possible to easily and conveniently upgrade the X-ray system having the old system specification of the related art, and it is possible to legally upgrade the X-ray system without illegal issues caused by additionally providing an extract line to the existing facility.

As stated above, according to the X-ray system of the present invention, since the operation switch includes the separate transmission means for generating and transmitting the signal, the signal is received by the speaker control unit simultaneously when the operation switch is operated. Thus, the voice signal for requesting patient's cooperation in the X-ray imaging can be output through the speaker under the control of the speaker control unit. Similarly, in the X-ray system including the DR controller, the DR controller operates the speaker control unit according to the switching operation of the operation switch, and thus, it is possible to output the voice signal for requesting patient's cooperation in the X-ray imaging through the speaker.

Accordingly, the patient can cooperate in the X-ray imaging in response to the output voice signal. Thus, since the operator does not need to open the door of the operation room in order to express his or her intention, it is possible to prevent the operator from being exposed to the X-rays when the operator operates the X-ray imaging.

### DESCRIPTION OF DRAWINGS

FIG. 1 is a schematic diagram for describing an X-ray system having an old system specification according to the related art.

FIG. 2 is a schematic diagram for describing an X-ray system having a new system specification according to the related art.

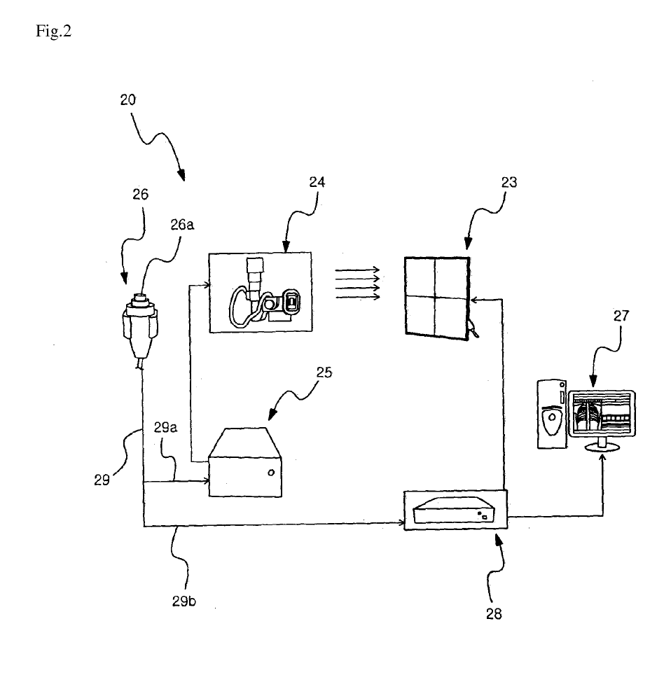

FIG. 3 is a plan view of an X-ray room where the X-ray system of FIG. 1 or FIG. 2 is provided.

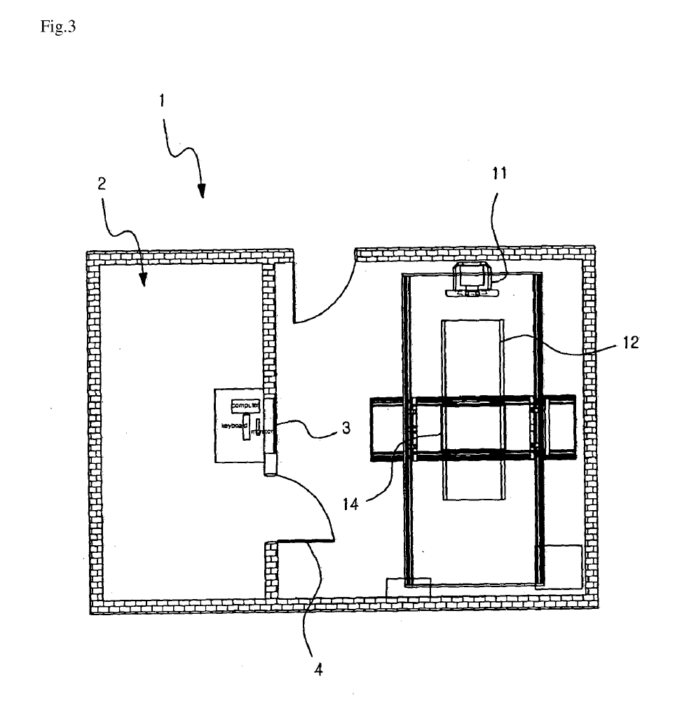

FIG. 4 is a schematic diagram of an operation switch for an X-ray system according to a first embodiment of the present invention.

FIG. 5 is a partial schematic diagram for describing an X-ray system including the operation switch of FIG. 4.

FIG. 6 is a partial schematic diagram for describing a modification example of the X-ray system of FIG. 5.

FIG. 7 is a schematic diagram of an operation switch for an X-ray system according to a second embodiment of the present invention.

FIG. 8 is a schematic diagram for describing an internal configuration of the operation switch of FIG. 7.

FIG. 9 is a plan view of an X-ray room where an X-ray system including the operation switch of FIG. 4 is provided.

FIG. 10 is a schematic diagram of an X-ray system according to another embodiment of the present invention.

### BEST MODE

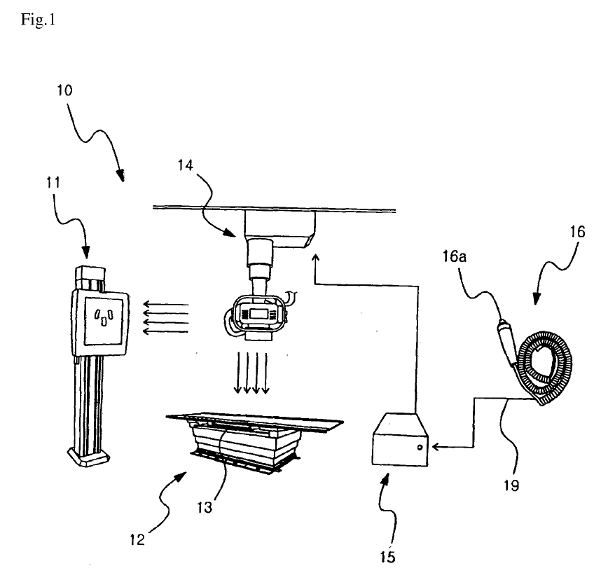

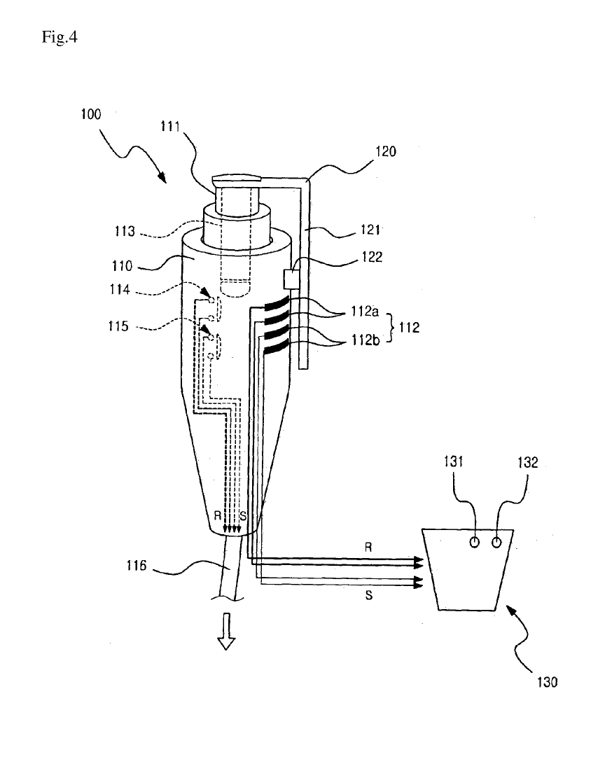

An X-ray system according to embodiments of the present invention basically includes the configuration of the X-ray system (see reference numeral 10) having the old system specification of the related art shown in FIG. 1, and includes an improved operation switch 100 shown in FIG. 4 as the operation switch which is one component thereof.

The operation switch 100 includes a button part 111 at an upper end of a switch body 110 as in the related art.

In addition, the operation switch 100 includes a switching terminal 112 attached onto an outer surface of the switch body 110.

The button part 111 is integrally connected to a link part 120 through an upper end surface thereof.

The link part 120 is formed to extend from the button part 111 in horizontal and vertical directions, and a vertical part 121 is provided so as to slide on the outer surface of the switch body 110 in a vertical axial direction.

Since a switching protrusion 122 is provided on an inner surface of the vertical part 121, the switching protrusion 122 slides downward on the outer surface of the switch body 110 when the button part 111 is pressed, and is in contact with the switching terminal.

In general, a pressing operation of the button part 111 of the operation switch 110 is distinguished between a first pressing operation and a second pressing operation for sequentially operating a generator (see reference numeral 15 of FIG. 1) in order to achieve preliminary driving for a preparation operation of an X-ray tube (see reference numeral 14 of FIG. 1) and main driving for emitting X-rays.

Correspondingly, the switching terminal 112 includes a first terminal 112a, and a second terminal 112b that are vertically arranged in parallel so as to correspond to the preliminary driving and the main driving of the X-ray tube.

Accordingly, the switching protrusion 122 is in contact with the first terminal 112a at the time of the first pressing operation of the button part 111 is performed, and is in contact with the second terminal 112b at the time of the second pressing operation of the button part 111.

Meanwhile, the operation switch 100 further includes a wireless transmitter 130 in order to generate a wireless signal by bringing the switching protrusion 122 into contact with the switching terminal 112.

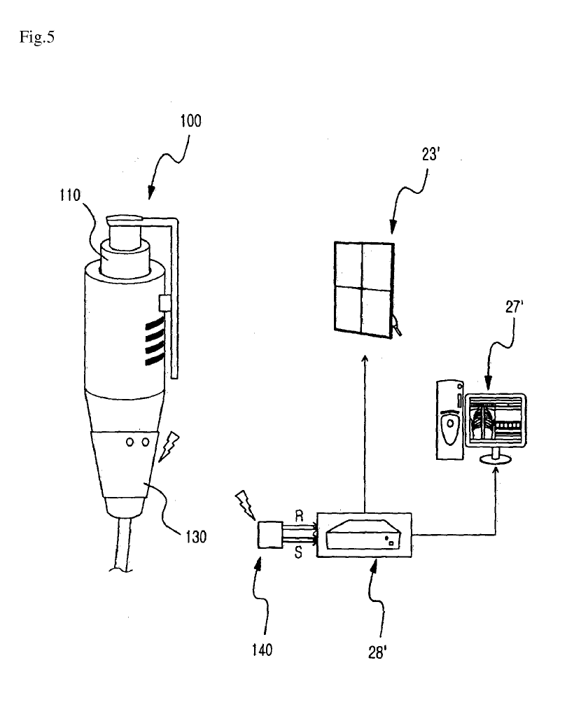

The wireless transmitter 130 is built in or attached to the switch body 110. In the present embodiment, the wireless transmitter is formed in a cone shape, and is fastened by being fitted to a lower end of the switch body 110 as shown in FIG. 5.

The wireless transmitter 130 is electrically connected to the switching terminal 112, and generates a first wireless signal when the switching protrusion 122 is in contact with the first terminal 112a and generates a second wireless signal when the switching protrusion 122 is in contact with the second terminal 112b.

Here, the first wireless signal is a signal for allowing a DR controller (see reference numeral 28′ of FIG. 5), to be described below, to perform the preliminary driving, as a preliminary driving signal R, and the second wireless signal is a signal for allowing the DR controller 28′ to perform the main driving, as a main driving signal S.

Of course, the operation switch 100 is in contact with a first switch 114 by a pin 113 provided within the switch body 110 at the time of the first pressing operation of the button part 111, and thus, the generator (see reference numeral 15 of FIG. 1) that is electrically connected through an electric wire 116 performs the preliminary driving. The operation switch is in contact with a second switch 115 by the pin 113 at the time of the second pressing operation of the button part 111, and thus, the generator 15 performs the main driving.

The X-ray tube (see reference numeral 14 of FIG. 1) starts the preliminary driving for emitting the X-rays by the preliminary driving signal of the generator 15, and the X-ray tube 14 emits and applies the X-rays by the main driving signal of the generator 15.

In this case, the first and second wireless signals generated by the wireless transmitter 130 are received by a wireless receiver 140 provided at the DR controller 28′, as shown in FIG. 5.

The wireless receiver 140 may be provided by being built in the DR controller 28′ or coming in contact with an external contact terminal.

The first and second wireless signals received by the wireless receiver 140 are received by the DR controller 28′, as the preliminary driving signal R and the main driving signal S, and the DR controller 28′ allows a DR detector 23′ to perform the preliminary driving or the main driving in response to the received signals. That is, the driving control is performed in synchronization with the generator 15.

The DR detector 23′ performs a preliminary operation in response to the preliminary driving signal of the DR controller 28′, and performs a main operation for receiving the X-rays in response to the main driving signal.

The X-rays received by the main operation of the DR detector 23′ are immediately displayed on an image processing and displaying device 27′ through the DR controller 28′.

Meanwhile, in FIG. 4 or FIG. 5, the wireless transmitter 130 may include a first lamp 131 that is turned on when the first wireless signal is generated, and a second lamp 132 that is turned on when the second wireless signal is generated, and may notify that each signal is generated to the outside.

Meanwhile, although it has been described in the embodiment that the button part 111 of the operation switch 100 performs the two pressing operations, the present invention is not limited thereto. In some cases, the present invention may be applied to a case where the button part 111 performs one pressing operation. In this case, the switching terminal 112 includes a single terminal (112a or 112b), and generates a wireless driving signal through the wireless transmitter 130 when the switch is turned on.

Although it has been described in the aforementioned embodiment that the wireless transmitter 130 and the wireless receiver 140 are wirelessly connected to each other, the present invention is not limited to such a wireless transmission method, and may be connected to each other in a wired manner when necessary.

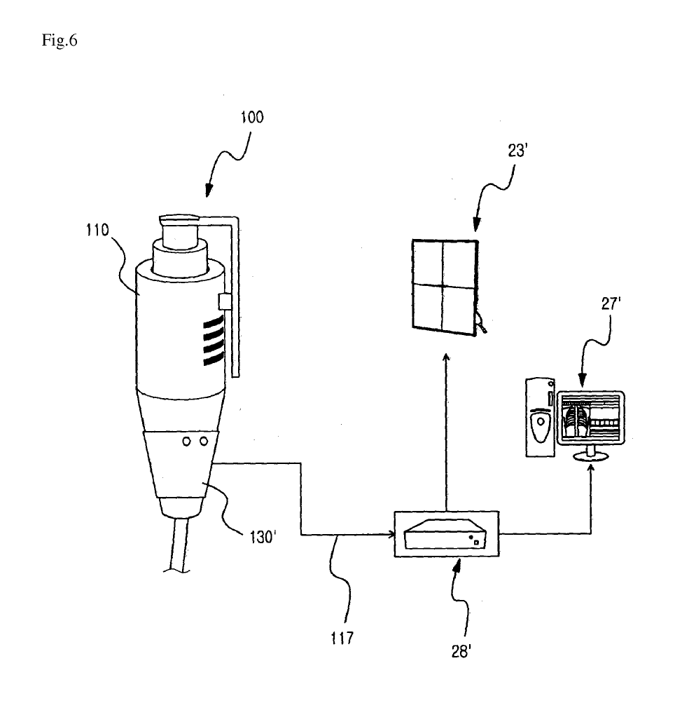

That is, a wired transmitter 130′ shown in FIG. 6 may be provided in place of the wireless transmitter 130 connected to the switching terminal 112 of the operation switch 100, and an electric wire 117 for transmitting the preliminary driving signal R and the main driving signal S generated in the wired transmitter may be directly connected to the DR controller 28′.

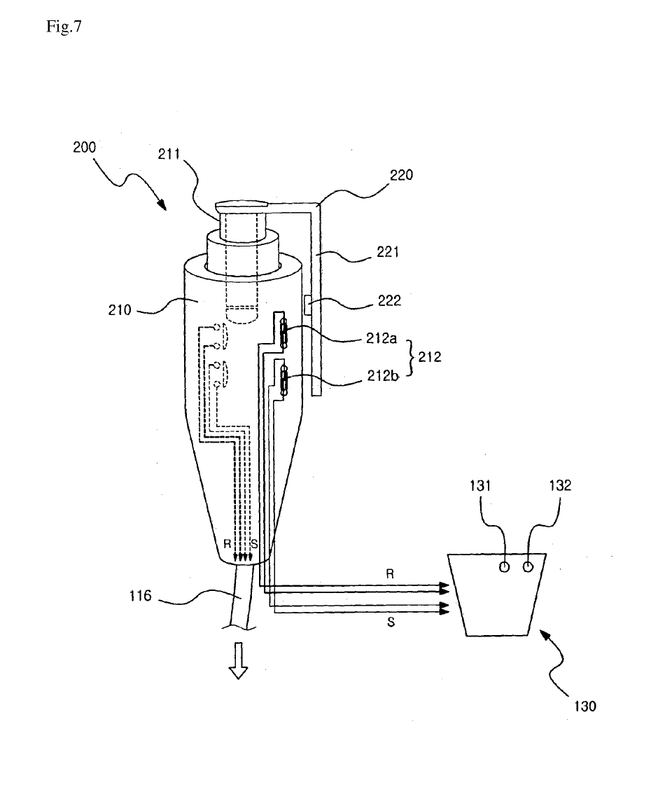

Meanwhile, as shown in FIG. 7, an operation switch 200 for an X-ray system according to a second embodiment of the present invention includes a lead switch 212, and a magnet 222, as switching means.

The lead switch 212 is provided by being fixed to the inside of the switch body 210, and the magnet 222 is provided by being attached onto an inner surface of a vertical part 221 of a link part 220. Thus, the magnet vertically slides on an outer surface of a switch body 210, and thus, the lead switch 212 is operated.

In this case, the lead switch 212 includes a first lead switch 212a that is operated at the time of a first pressing operation of the button part 211, and a second lead switch 212b that is operated at the time of a second pressing operation of the button part 211. Correspondingly, the wireless transmitter 130 distinguishingly generates a first signal and a second signal.

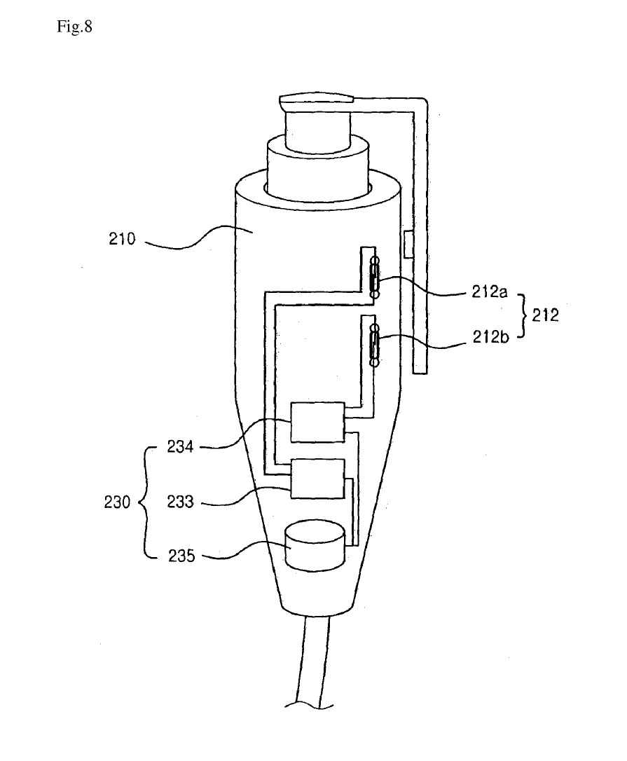

Meanwhile, in the first and second embodiments, the wireless transmitter 130 and the wired transmitter 130′ may be provided by being fitted to the lower end of the switch body 110 as shown in FIGS. 5 and 6, or may be provided by being built in the switch body 210 as shown in FIG. 8.

In FIG. 8, the wireless transmitter 230 includes a first wireless transmitter 233 for generating a first wireless signal, and a second wireless transmitter 234 for generating a second wireless signal, and these transmitters are electrically connected to the first lead switch 212a and the second lead switch 212b. The wireless transmitter further includes a battery 235 for supplying power to these wireless transmitters 233 and 234.

In order to replace the battery 235 with a new one, a structure in which a part of the lower end of the switch body 210 is opened may be provided.

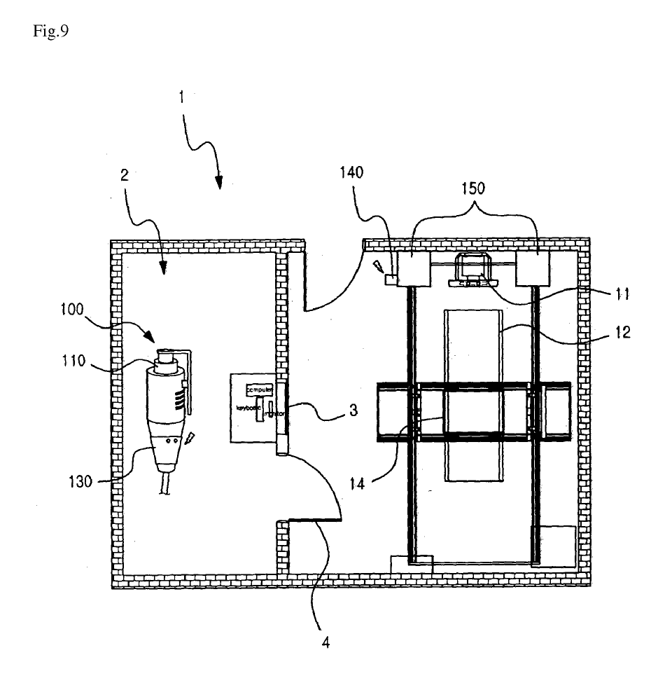

Meanwhile, the first and second wireless signals generated by the wireless transmitter 130 shown in FIG. 4 are received by a speaker control unit 140 disposed adjacent to an X-ray stand 11 or an X-ray bed 12 provided within an X-ray room 1 as shown in FIG. 9.

Similarly, the speaker control unit 140 may be provided by being attached to a pair of speakers 150 arranged adjacent to the X-ray stand 11 or the X-ray bed 12.

As described above, the first and second wireless signals of the operation switch 100 are received by the generator 15, as the preliminary driving signal R and the main driving signal S, and thus, the generator 15 is synchronized. Accordingly, the preliminary driving control and the main driving control may be performed.

In this case, the speaker control unit 140 that is synchronized with the generator 15 to receive the first and second wireless signals performs control such that a voice signal for requesting patient's cooperation in X-ray imaging, for example, a voice signal for requesting preparation for the X-ray imaging, such as “inhale and hold your breath, please”, is output through the speakers 150 when the first wireless signal is received. The speaker control unit performs control such that a voice signal for notifying that the X-ray imaging is completed, for example, a voice signal such as “X-ray imaging is completed” is output when the second wireless signal is received.

Accordingly, since an operator can request patient's cooperation even when a door 4 of an operation room 2 is closed as in the related art, it is possible to solve a problem about X-ray exposure caused by opening the door 4 of the operation room.

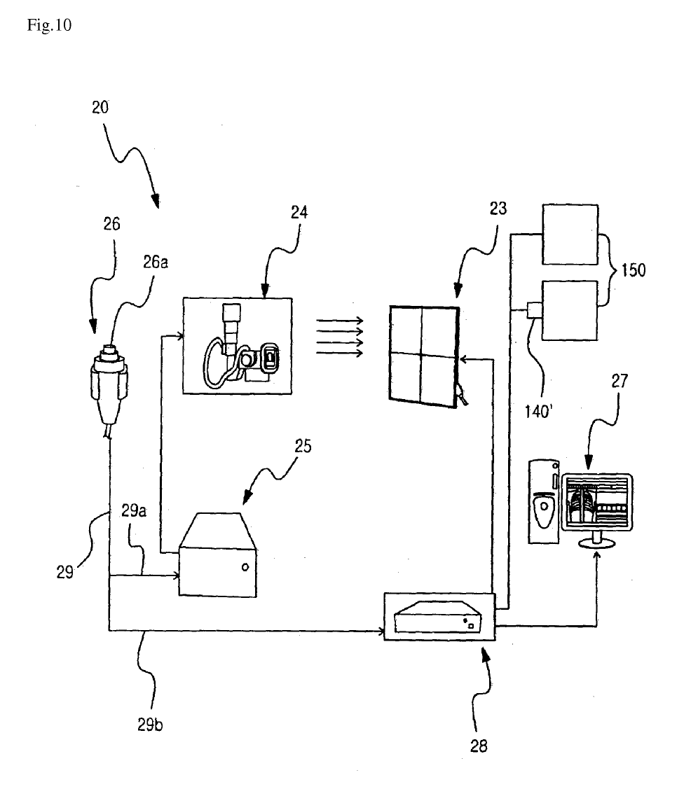

As shown in FIG. 10, an X-ray system 20 according to another embodiment of the present invention is an X-ray system 20 having a new specification using a DR cassette of the related art, and includes an X-ray reception cassette 23 which is a DR detector attached to the X-ray stand (see reference numeral 11 of FIG. 1) or the X-ray bed (see reference numeral 12 of FIG. 1), an X-ray tube 24 for emitting X-rays to the X-ray reception cassette 23, a generator 25 for driving the X-ray tube, an operation switch 26 provided with a button part 26a for operating the generator, an image processing and displaying device 27 for displaying reception data of the X-ray reception cassette as an image, and a DR controller 28 for controlling the X-ray reception cassette and the image processing and displaying device.

As in the aforementioned embodiment, the X-ray system 20 further includes a speaker control unit 140′, and a speaker 150′.

In the present embodiment, the button part 26a of the operation switch 26 performs a first pressing operation and a second pressing operation for sequentially operating the generator 25 in order to achieve preliminary driving for a preparation operation of the X-ray tube 24 and main driving for emitting the X-rays. The DR controller 28 generates a first signal by the first pressing operation of the button part 26a, and generates a second signal in response to the second pressing operation of the button part 26a.

The first signal and the second signal of the DR controller 28 are transmitted to the speaker control unit 140′ connected to the DR controller in a wired manner. When the first signal is received, the speaker control unit 140′ may perform control such that a voice signal for requesting preparation for X-ray imaging is output through the speaker 150′, and when the second signal is received, the speaker control unit may perform control such that a voice signal for notifying that the X-ray imaging is completed is output through the speaker 150′.

Meanwhile, although it has been described in the embodiments that only wireless communication (first embodiment) or wired communication (second embodiment) is applied to the speaker control units 140 and 140′, the present invention is not limited to such a transmission method, and the any one of wired communication and wireless communication may be used when necessary.

Meanwhile, the operation switch 100 or 200 for the X-ray system and the X-ray system 10 or 20 described above are merely examples for helping the understanding the present invention, and it should be understood that the right scope and technical scope of the present invention are not intended to be limited to the aforementioned description.

The right scope and technical scope of the present invention are defined by the claims and their equivalents to be described below.

1. An X-ray system that includes an X-ray reception cassette which is attached to an X-ray stand or an X-ray bed, an X-ray tube which emits X-rays to the X-ray reception cassette, a generator which drives the X-ray tube, an operation switch that is provided with a button part for operating the generator, an image processing and displaying device which displays reception data of the X-ray reception cassette as an image, and a controller which controls the X-ray reception cassette and the image processing and displaying device,
wherein the operation switch includes
a switch body,
a link part that is integrally formed with the button part so as to extend, and slides on an outer surface of the switch body at the time of pressing the button part,
switching means for performing a switching operation according to the sliding of the link part, and
transmission means for generating a signal according to the switching operation of the switching means, and for transmitting the generated signal to the controller, and
wherein the controller performs control such that the X-ray reception cassette is turned on when the signal is received.

2. The X-ray system of claim 1,
wherein the transmission means is wireless transmission means, and generates a wireless signal, and
the controller includes wireless reception means for receiving the wireless signal.

3. The X-ray system of claim 1,
wherein the transmission means is wired transmission means, and generates a wired signal, and
the controller receives the wired signal through an electric wire that is electrically connected to the wired transmission means.

4. The X-ray system of claim 1,
wherein the switching means includes a switching terminal that is provided on the outer surface of the switch body, and a switching protrusion that is provided on the link part, and is in contact with the switching terminal at the time of sliding on the outer surface of the switch body.

5. The X-ray system of claim 4,
wherein the button part performs a first pressing operation and a second pressing operation for sequentially operating the generator in order to achieve preliminary driving for a preparation operation of the X-ray tube and main driving for emitting X-rays,
the switching terminal includes a first terminal, and a second terminal which are arranged in parallel so as to correspond to the preliminary driving and the main driving of the X-ray tube,
the switching protrusion is in contact with the first terminal at the time of the first pressing operation of the button part, and is contact with the second terminal at the time of the second pressing operation of the button part,
the transmission means generates a first signal when the switching protrusion is in contact with the first terminal, and generates a second signal when the switching protrusion is in contact with the second terminal, and
the controller performs control such that the X-ray reception cassette performs the preliminary driving when the first signal is received, and performs control such that the X-ray reception cassette performs the main driving when the second signal is received.

6. The X-ray system of claim 5,
wherein the transmission means includes first alarm means that is turned on when the first signal is generated, and second alarm means that is turned on when the second signal is generated.

7. The X-ray system of claim 1,
wherein the switching means includes a lead switch that is provided inside the switch body, and a magnet that is provided at the link part, and operates the lead switch at the time of sliding on the outer surface of the switch body.

8. The X-ray system of claim 7,
wherein the button part performs a first pressing operation and a second pressing operation for sequentially operating the generator in order to achieve preliminary driving for a preparation operation of the X-ray tube and main driving for emitting X-rays,
the lead switch includes a first lead switch and a second lead switch that are arranged in parallel so as to correspond to the preliminary driving and the main driving of the X-ray tube,
the magnet operates the first lead switch at the time of the first pressing operation of the button part, and operates the second lead switch at the time of the second pressing operation of the button part,
the transmission means generates the first signal when the first lead switch is operated, and generates the second signal when the second lead switch operates, and
the controller performs control such that the X-ray reception cassette performs the preliminary driving when the first signal is received, and performs control such that the X-ray reception cassette performs the main driving when the second signal is received.

9. The X-ray system of claim 8,
wherein the transmission means includes first alarm means that is turned on when the first signal is generated, and second alarm means that is turned on when the second signal is generated.

10. An operation switch for an X-ray system comprising:
a switch body that is provided with a button part at one end thereof;
a link part that is integrally formed with the button part so as to extend, and slides on an outer surface of the switch body when the button part is pressed;
switching means that is switched according to the sliding of the link part; and
transmission means for generating a signal according to the switching operation of the switching means, and for transmitting the generated signal.

11. The operation switch for an X-ray system of claim 10,
wherein the switching means includes a switching terminal that is provided on the outer surface of the switch body, and a switching protrusion that is provided at the link part, and is in contact with the switching terminal at the time of sliding on the outer surface of the switch body.

12. The operation switch for an X-ray system of claim 10,
wherein the switching means includes a lead switch that is provided inside the switch body, and a magnet that is provided at the link part, and operates the lead switch at the time of sliding on the outer surface of the switch body.

13. The X-ray system of claim 1, further comprising:
a speaker control unit that is operated by receiving a signal of the transmission means; and
a speaker that is disposed adjacent to the X-ray stand or the X-ray bed, and outputs a voice signal for requesting patient's cooperation in X-ray imaging under the control of the speaker control unit.

14. The X-ray system of claim 13,
wherein the transmission means is wired transmission means, and generates a wired signal, and
the speaker control unit receives the wired signal through an electric wire that is electrically connected to the wired transmission means.

15. The X-ray system of claim 13,
wherein the switching means includes a switching terminal that is provided on the outer surface of the switch body, and a switching protrusion that is provided at the link part, and is in contact with the switching terminal at the time of sliding on the outer surface of the switch body,
the button part performs a first pressing operation and a second pressing operation for sequentially operating the generator in order to achieve preliminary driving for a preparation operation of the X-ray tube and main driving for emitting X-rays,
the switching terminal includes a first terminal, and a second terminal which are arranged in parallel so as to correspond to the preliminary driving and the main driving of the X-ray tube,
the switching protrusion is in contact with the first terminal at the time of the first pressing operation of the button part, and is contact with the second terminal at the time of the second pressing operation of the button part,
the transmission means generates a first signal when the switching protrusion is in contact with the first terminal, and generates a second signal when the switching protrusion is in contact with the second terminal, and
the speaker control unit performs control such that a voice signal for requesting preparation for X-ray imaging is output through the speaker when the first signal is received, and performs control such that a voice signal for notifying that the X-ray imaging is completed is output through the speaker when the second signal is received.

16. The X-ray system of claim 13,
wherein the switching means includes a lead switch that is provided inside the switch body, and a magnet that is provided at the link part, and operates the lead switch at the time of sliding on the outer surface of the switch body,
the button part performs a first pressing operation and a second pressing operation for sequentially operating the generator in order to achieve preliminary driving for a preparation operation of the X-ray tube and main driving for emitting X-rays,
the lead switch includes a first lead switch and a second lead switch that are arranged in parallel so as to correspond to the preliminary driving and the main driving of the X-ray tube,
the magnet operates the first lead switch at the time of the first pressing operation of the button part, and operates the second lead switch at the time of the second pressing operation of the button part,
the transmission means generates a first signal when the first lead switch is operated, and generates a second signal when the second lead switch is operated, and
the speaker control unit performs control such that a voice signal for requesting preparation for X-ray imaging is output through the speaker when the first signal is received, and performs control such that a voice signal for notifying that the X-ray imaging is completed is output through the speaker when the second signal is received.

17. An X-ray system that includes an X-ray reception cassette which is a DR detector attached to an X-ray stand or an X-ray bed, an X-ray tube which emits X-rays to the X-ray reception cassette, a generator which drives the X-ray tube, an operation switch which is provided with a button part for operating the generator, an image processing and displaying device which displays reception data of the X-ray reception cassette as an image, and a DR controller which controls the X-ray reception cassette and the image processing and displaying device, the DR controller performing control such that the X-ray reception cassette is turned on when a signal generated according to the switching operation of the operation switch is received, the X-ray system comprising:
a speaker control unit that is operated by the DR controller by turning on the X-ray reception cassette; and
a speaker that is disposed adjacent to the X-ray stand or the X-ray bed, and outputs a voice signal for requesting patient's cooperation in X-ray imaging under the control of the speaker control unit.

18. The X-ray system of claim 17,
wherein the button part performs a first pressing operation and a second pressing operation for sequentially operating the generator in order to achieve preliminary driving for a preparation operation of the X-ray tube and main driving for emitting X-rays,
the DR controller generates a first signal according to the first pressing operation of the button part, and generates a second signal according to the second pressing operation of the button part, and
the speaker control unit performs controls such that a voice signal for requesting preparation for X-ray imaging is output through the speaker when the first signal is received, and performs controls such that a voice signal for notifying that the X-ray imaging is completed is output through the speaker when the second signal is received.
